(19) United States
(12) Patent Application Publication
(10) Pub. No.: US 2008/0175899 A1
(43) Pub. Date: Jul. 24, 2008

(54) FORMULATION FOR PROMOTING SINUS HEALTH IN NASAL CAVITIES

(76) Inventors: Mairi R. Ross, Jerome, AZ (US); Bob Cave, Lake Montezuma, AZ (US)

Correspondence Address: JAECKLE FLEISCHMANN & MUGEL, LLP, 190 Linden Oaks, ROCHESTER, NY, 14625-2812, US

(21) Appl. No.: 12/016,541

(22) Filed: Jan. 18, 2008

Related U.S. Application Data
(60) Provisional application No. 60/885,655, filed on Jan. 19, 2007.

(51) Int. Cl. A61K 9/48; A61K 31/353; A61K 35/74; A61P 11/02; A61K 9/28

(52) U.S. Cl. 424/453; 514/456; 424/93.4; 424/93.45; 424/93.44; 424/474; 424/93.3

(57) ABSTRACT

A formulation for promoting sinus health includes at least one probiotic agent in combination with N-acetyl cysteine (NAC) and a bioflavonoid or derivative thereof.

### CROSS-REFERENCE TO RELATED APPLICATIONS

This application claims the benefit of U.S. Provisional Patent Application Ser. No. 60/885,655, filed Jan. 19, 2007.

### FIELD OF THE INVENTION

The present invention relates to the promotion of nasal sinus health and, more particularly, to a formulation for clearing potentially pathogenic bacteria and for reducing excess mucus and inflammation in nasal cavities.

### BACKGROUND OF THE INVENTION

Three of the major risk factors for nasal sinus health are: the presence of potentially pathogenic bacteria (PPB) in the nasal cavities, which can lead to sinusitis, pneumonia, or otitis; the build up of thick mucus in the nasal cavities, which would provide a breeding ground for viruses, infections, and fungi; and swelling and inflammation resulting from poor sinus drainage, allergies, or infections.

Probiotics, which may be defined as live microorganisms that confer a beneficial health effect on a host, represent a potentially useful alternative to antibiotics or antiseptics for controlling PPB in the nasal cavity. Typically, probiotics are bacteria of, for example, the genera Lactobacillus and Bifidobacterium. Such “good” bacteria may possibly function by affecting the mucosal immune response and displacing “bad” PPB, thereby helping to maintain a healthy balance of good flora in the nasal cavities.

The inclusion of probiotics as additives in food, dietary supplements, and medical products is well known, for example: a milk drink fermented with high levels of specific probiotics which, when ingested, is reported to reduce nasal colonization of pathogenic bacteria (Gluck and Gebbers, Am. J. Clin. Nutr, 2003); dietary yoghurt (Aldinucci, Eur. J Clin. Nutr, 2002); New Chapter Sinus and Respiratory, containing two billion cells of probiotics cultured in a base of elderberry, spinach, parsley, okra, oregano, cumin, ginger, arabinogalactan, milk and nutritional yeast. Also, probiotic bacteria such as Lactobacillus acidophilus are sold as gel capsules containing a lyophilized mixture of bacterial cells and a solid support such as mannitol; upon ingestion with liquid, the lyophilized cells are re-hydrated and become viable bacteria.

Normal mucus is moderately thin and drains freely from the nasal cavities. If it becomes too thick it may clog the nasal cavities, which can become a breeding ground for bacteria, fungi, or viruses. N-acetylcysteine (NAC), is a pharmacological agent that is frequently used in mucolytic (“mucus dissolving”) therapy. NAC acts to reduce mucus viscosity, possibly by splitting disulfide bonds linking proteins present in the mucus (mucoproteins). It is generally formulated as a solution that is administered by nasal inhalation. A NAC-containing formulation for this purpose is available under the trade name Mucomyst™ from Bristol-Myers Squibb.

A commercially available expectorant formulation, Natural Factors Dr. Murray's Lung Bronchial & Sinus Health tablets, includes NAC together with bromelain, marshmallow, mullein, wild cherry, and horehound.

U.S. Pat. No. 4,847,282, the disclosure of which is incorporated herein by reference, describes water-soluble N-acetylcysteine salts that are formed by the reaction of N-acetylcysteine with a basic amino acid selected from among arginine, lysine, histidine, ornithine, and glycine.

U.S. Patent Appl. Serial No. 2005/0154060, the disclosure of which is incorporated herein by reference, describes the use of formulations of water-soluble N-acetylcysteine salts to be administered by inhalation directly to the lungs and/or nose.

International Application WO 98/48839, the disclosure of which is incorporated herein by reference, describes nasally administered compositions comprising a topical anti-inflammatory agent and at least one agent selected from a group that includes an antihistamine and a mucolytic agent.

Bioflavonoids and derivatives thereof constitute a group of naturally occurring compounds that are widely distributed among plants, including most citrus fruits, rose hips, and black currants. A wide variety of physiological effects have been attributed to bioflavonoids, including the maintenance of normal blood vessel conditions by decreasing capillary permeability and fragility, and activity as a histamine release blocker for the treatment of allergies.

Quercetin, a bioflavonoid that forms the backbone for many other bioflavonoid derivatives such as the citrus bioflavonoids rutin and hesperidin, occurs naturally in onions, apples, green tea, and black tea and, in lesser amounts, in leafy green vegetables and beans. Quercetin has demonstrated significant anti-inflammatory activity resulting from direct inhibition of several initial processes of inflammation, for example, the inhibition of both the production and the release of histamine and other allergic/inflammatory mediators. Owing to its antihistamine and anti-inflammatory effects, it is useful for reducing inflammation in the nasal cavities, especially inflammation resulting from allergies.

There is a need for a convenient, orally administered composition for promoting nasal sinus health that contains an effective dosage of a plurality of agents that counteract several of the major risk factors for nasal sinus health.

### SUMMARY OF THE INVENTION

The present invention is directed to a formulation for promoting sinus health that comprises at least one probiotic agent in combination with N-acetyl cysteine (NAC) and a bioflavonoid or derivative thereof.

### DETAILED DESCRIPTION OF THE INVENTION

Continuous infections and inflammation of the sinus mucosa can block drainage, trap mucus and create a breeding ground for bacteria and viruses. In some cases of prolonged inflammation, the mucosal lining may swell into polyps, which further obstructs drainage of mucus and inhibits air flow. Breaking up the clogged mucus helps to prevent the growth of pathogenic bacteria, fungi or viruses and thus helps reduce swelling and inflammation.

The formulation of the present invention includes effective doses of the following components: a probiotic agent that reduces PPB and thereby provides a healthy balance of good bacteria, the mucolytic agent N-acetylcysteine (NAC) for aiding the break up of excess mucus in the nasal cavities, and a bioflavonoid, or derivative thereof. A preferred bioflavonoid is quercetin, an antihistamine and anti-inflammatory agent that acts to reduce swelling and inflammation. The combination of these ingredients in the formulation of the invention enables three major risk factors for sinus health to be addressed substantially simultaneously.

Unlike available liquid fermented milk or yoghurt products that are inconvenient to transport and use, which would be less convenient to use, the formulation of the present invention is preferably in a solid form, for example, a powder, a tablet, a capsule, a caplet, an encapsulated liquid, and the like.

Probiotic agents preferably include bacteria such as, for example, Lactobacillus acidophilus, Lactobacillus casei, Lactobacillus plantarum, Lactobacillus reuteri, Lactobacillus rhamnosus, Lactobacillus GG, Bifidobacterium bifidum, Bifidobacterium breve, Bifidobacterium infantis, and Bifidobacterium longum, Streptococcus thermophilus and mixtures thereof. An effective dosage of a probiotic agent preferably comprises about 1 billion bacteria to about 100 billion bacteria, more preferably, about 15 billion bacteria to about 50 billion bacteria.

N-acetylcysteine has limited aqueous solubility. Where increased water-solubility of the mucolytic agent in the formulation of the present invention is desired, a water-soluble salt of N-acetylcysteine may be employed, as disclosed in the previously discussed U.S. Pat. No. 4,847,282.

The bioflavonoid quercetin has limited aqueous solubility, but chalcone derivatives of bioflavonoids are typically readily soluble in water. U.S. Pat. No. 5,977,184, the disclosure of which is incorporated herein by reference, describes the preparation of quercetin chalcone, which may be employed in the composition of the present invention when improved water solubility of the bioflavonoid component is desired.

In the formulation of the present invention, an effective dosage of N-acetylcysteine (NAC) preferably comprises about 100 milligrams to about 1200 milligrams, more preferably, about 200 milligrams to about 1000 milligrams. A preferred formulation of the invention further comprises an effective dosage of quercetin, preferably about 100 milligrams to about 1000 milligrams, more preferably, about 200 milligrams to about 600 milligrams.

Preferably, the solid composition of the present invention comprises a tablet or two-piece hard shell capsule, along with an appropriate amount of at least one formulating agent, which is required to put the composition in tablet or capsule form. Such agents, which may be present in the composition in a total amount of up to about 95 weight percent, include, for example, fillers, flow agents, colorants, flavorings, dissolving agents, and the like, all well known in the art.

In one embodiment, the composition of the present invention is in the form of a tablet. In one embodiment, the tablet comprises an enteric coating. In another embodiment, the tablet may be constituted in a chewable form. Alternatively, the tablet may be formulated to effervesce when dissolved in water.

In a further embodiment, the composition of the invention is in the form of a two-piece hard shell capsule, in which a flow agent is particularly useful. Preferred flow agents include magnesium stearate, silica, and mixtures thereof.

In still a further embodiment, the composition is in the form of an encapsulated liquid.

While the invention has been described by reference to various specific embodiments, it should be understood that numerous changes may be made within the spirit and scope of the inventive concepts described. Accordingly, it is intended that the invention not be limited to the described embodiments, but will have full scope defined by the language of the following claims.

What is claimed:

1. An orally administered composition for promoting nasal sinus health comprising at least one probiotic agent in combination with N-acetylcysteine (NAC) and a bioflavonoid or derivative thereof.

2. The composition of claim 1 comprising an effective dosage of each of said probiotic agent, N-acetylcysteine (NAC), and bioflavonoid or derivative thereof.

3. The composition of claim 1 wherein said bioflavonoid or derivative thereof comprises quercetin.

4. The composition of claim 1 wherein said probiotic agent comprises bacteria.

5. The composition of claim 4 wherein said bacteria are selected from the group consisting of Lactobacillus acidophilus, Lactobacillus casei, Lactobacillus plantarum, Lactobacillus reuteri, Lactobacillus rhamnosus, Lactobacillus GG, Bifidobacterium bifidum, Bifidobacterium breve, Bifidobacterium infantis, Bifidobacterium longum, Streptococcus thermophilus, and mixtures thereof.

6. The composition of claim 4 wherein said bacteria comprise an effective dosage of about 1 billion bacteria to about 100 billion bacteria.

7. The composition of claim 6 wherein said bacteria comprise an effective dosage of about 15 billion bacteria to about 50 billion bacteria.

8. The composition of claim 2 wherein said effective dosage of N-acetylcysteine (NAC) comprises about 100 milligrams to about 1200 milligrams.

9. The composition of claim 8 wherein said effective dosage of N-acetylcysteine (NAC) comprises about 200 milligrams to about 1000 milligrams.

10. The composition of claim 1 wherein N-acetylcysteine (NAC) is incorporated as a water-soluble salt in said composition.

11. The composition of claim 2 wherein said effective dosage of said bioflavonoid or derivative thereof comprises about 100 milligrams to about 1000 milligrams of quercetin.

12. The composition of claim 11 wherein said effective dosage of bioflavonoid or derivative thereof comprises about 200 milligrams to about 600 milligrams of quercetin.

13. The composition of claim 1 comprising a solid form.

14. The composition of claim 13 wherein said solid form comprises a powder, a tablet, a capsule, a caplet, or an encapsulated liquid.

15. The composition of claim 1 further comprising at least one formulating agent.

16. The composition of claim 15 wherein said formulating agent is selected from the group consisting of a filler, a flow agent, a colorant, a flavoring, a dissolving agent, and mixtures thereof.

17. The composition of claim 16 wherein said formulating agent comprises up to about 95 weight percent of said composition.

18. The composition of claim 14 wherein said solid form comprises a tablet.

19. The composition of claim 18 wherein said tablet comprises an enteric coating.

20. The composition of claim 18 wherein said tablet is chewable.

21. The composition of claim 18 wherein said tablet effervesces upon dissolution in water.

22. The composition of claim 14 wherein said solid form comprises a capsule.

23. The composition of claim 22 wherein said capsule comprises a two-piece hard shell capsule.

24. The composition of claim 22 wherein said capsule comprises a flow agent.

25. The composition of claim 14 wherein said solid form comprises a powder.

26. The composition of claim 14 wherein said solid form comprises an encapsulated liquid.

27. An orally administered composition for promoting nasal sinus health comprising an effective dosage of each of a probiotic agent, N-acetylcysteine (NAC), and a bioflavonoid or derivative thereof, said effective dosage comprising about 1 billion probiotic bacteria to about 100 billion probiotic bacteria, in combination with about 100 milligrams to about 1200 milligrams N-acetylcysteine (NAC) and about 100 milligrams to about 1000 milligrams of the bioflavonoid quercetin.

28. The orally administered composition of claim 26, wherein said effective dosage comprises about 15 billion probiotic bacteria to about 50 billion probiotic bacteria, in combination with about 200 milligrams to about 1000 milligrams N-acetylcysteine (NAC) and about 200 milligrams to about 600 milligrams of the bioflavonoid quercetin.
